(19) United States
(12) Patent Application Publication
(10) Pub. No.: US 2018/0020917 A1
(43) Pub. Date: Jan. 25, 2018

(54) PHYSIOLOGICAL MONITORING DEVICE, PHYSIOLOGICAL MONITORING METHOD AND NON-TRANSITORY COMPUTER READABLE STORAGE MEDIUM FOR IMPLEMENTING THE PHYSIOLOGICAL MONITORING METHOD

(71) Applicant: HTC CORPORATION, TAOYUAN CITY (TW)

(72) Inventors: Keng-Chih LIN, TAOYUAN CITY (TW); Yen-Liang KUO, TAOYUAN CITY (TW); Chien-Chih CHEN, TAOYUAN CITY (TW); Chun-Yih WU, TAOYUAN CITY (TW); Chieh-Sen LEE, TAOYUAN CITY (TW)

(73) Assignee: HTC CORPORATION, TAOYUAN CITY (TW)

(21) Appl. No.: 15/616,044

(22) Filed: Jun. 7, 2017

(30) Foreign Application Priority Data
Jul. 20, 2016 (TW) 105122870

(51) Int. Cl. A61B 5/00; G08B 21/04; G06K 9/00; G06K 9/62; A61B 5/0205; G06F 19/00

(52) U.S. Cl. CPC A61B 5/0006; A61B 5/0022; A61B 5/0205; G06F 19/3406; G06F 19/3418; G06K 9/00348; G06K 9/6201; G08B 21/0423

(57) ABSTRACT

A physiological monitoring device, a physiological monitoring method and a non-transitory computer readable storage medium for implementing the physiological monitoring method are provided. The physiological monitoring device has a blood pressure sensing module, a motion sensing module and a processor. The motion sensing module senses a physical state of the physiological monitoring device to generate a physical state signal accordingly. The processor, which is coupled to the blood pressure sensing module and the motion sensor module, determines whether the physiological monitoring device is in a stationary state and a horizontal state according to the physical state signal so as to determine whether an ideal measurement condition is met. When the ideal measurement condition is met, the processor controls the blood pressure sensing module to measure a blood pressure of the user.

### CROSS-REFERENCES TO RELATED APPLICATIONS

This application claims priority to Taiwan Patent Application No. 105122870 filed on Jul. 20, 2016, which is hereby incorporated by reference in its entirety.

### BACKGROUND OF THE INVENTION

### Field of the Invention

The present invention relates to a physiological monitoring device, a physiological monitoring method and a non-transitory computer readable storage medium thereof. More particularly, the physiological monitoring device of the present invention can monitor the physiological state/physiological activity of a user to measure a blood pressure of the user accordingly.

### Descriptions of the Related Art

Over recent years, various types of wearable devices (e.g., smart bracelets/bands, smart watches, heart rate belts, etc) that assist users in tracking his/her physiological state (e.g., various information on blood pressure, heart rate, body temperature, or sleep quality, etc) at any time have been widely developed.

Although the blood pressure of a user can be measured by a wearable device, the user is not always in an ideal state for measuring blood pressure. This is especially the case when the user is exercising or recuperating from strenuous exercise, raising his or her arm, or experiencing elevated emotions which would result in an inaccurate blood pressure measurement. Accordingly, it is important to provide a physiological monitoring mechanism that starts measuring blood pressure only when the user is in an ideal measurement state.

### SUMMARY OF THE INVENTION

An objective of the present invention is to provide a physiological monitoring mechanism that can detect the physiological state/physiological activity of a user and appropriately control the blood pressure sensing module to measure a blood pressure of the user according to the detected physiological state/physiological activity. As a result, the blood pressure of the user can be measured by the physiological monitoring mechanism of the present invention at an appropriate time, thus improving the reference value of the blood pressure measurement.

To achieve the aforesaid objective, a physiological monitoring device for monitoring the physiological state of a user is disclosed. The physiological monitoring device comprises a blood pressure sensing module, a motion sensing module and a processor. The motion sensing module is configured to sense the physical state of the physiological monitoring device to generate a physical state signal. The processor, which is coupled to the blood pressure sensing module and the motion sensor module, is configured to determine whether the physiological monitoring device is in a stationary state and a horizontal sate according to the physical state signal to determine whether an ideal measurement condition is met, and to control the blood pressure sensing module to measure a blood pressure of the user when the ideal measurement condition is met.

In addition, a physiological monitoring method for a physiological monitoring device is further disclosed. The method is applied to an electronic apparatus and comprises the following steps of: (a) receiving a physical state signal corresponding to the electronic apparatus; (b) determining whether the electronic apparatus is in a stationary state and a horizontal state according to the physical state signal to determine whether an ideal measurement condition is met; and (c) controlling the electronic apparatus to measure a blood pressure of a user when the ideal measurement condition is met.

Moreover, a non-transitory computer readable storage medium with a computer program stored therein is further disclosed. The computer program is loaded into an electronic apparatus to execute the following steps of: (a) receiving a physical state signal corresponding to the electronic apparatus; (b) determining whether the physiological monitoring device is in a stationary state and a horizontal state according to the physical state signal to determine whether an ideal measurement condition is met; and (c) controlling a blood pressure sensing module to measure a blood pressure of a user when the ideal measurement condition is met.

The detailed technology and preferred exemplary embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a block diagram of the hardware infrastructure of a physiological monitoring device in accordance with an exemplary embodiment of the present invention;

FIG. 2 is a block diagram of the hardware infrastructure of a physiological monitoring device comprising a heart rate sensing module in accordance with an exemplary embodiment of the present invention;

FIG. 3 is a block diagram of the hardware infrastructure of a physiological monitoring device comprising a temperature sensing module in accordance with an exemplary embodiment of the present invention;

FIG. 4 is a block diagram of the hardware infrastructure of a physiological monitoring device comprising a heart rate sensing module and a temperature sensing module in accordance with an exemplary embodiment of the present invention;

FIG. 5 is a flowchart diagram of a physiological monitoring method in accordance with an exemplary embodiment of the present invention;

FIG. 6 is a flowchart diagram of a physiological monitoring method in accordance with an exemplary embodiment of the present invention;

FIG. 7 is a flowchart diagram of a physiological monitoring method in accordance with an exemplary embodiment of the present invention; and

FIG. 8 is a flowchart diagram of a physiological monitoring method in accordance with an exemplary embodiment of the present invention.

### DESCRIPTION OF THE PREFERRED EMBODIMENT

In the following description, the present invention will be explained with reference to exemplary embodiments thereof The present invention relates to a physiological monitoring device, a physiological monitoring method and a non-transitory computer readable storage medium thereof. It shall be appreciated that these exemplary embodiments of the present invention are not intended to limit the present invention to any specific environment, applications or particular implementations described in these exemplary embodiments. Therefore, the description of these exemplary embodiments is only for the purpose of illustration rather than limitation and the scope of this application shall be governed by the claims. In the following exemplary embodiments and attached drawings, elements unrelated to the present invention are omitted from depiction; and dimensional relationships among individual elements in the attached drawings are illustrated only for ease of understanding, but not to limit the actual scale.

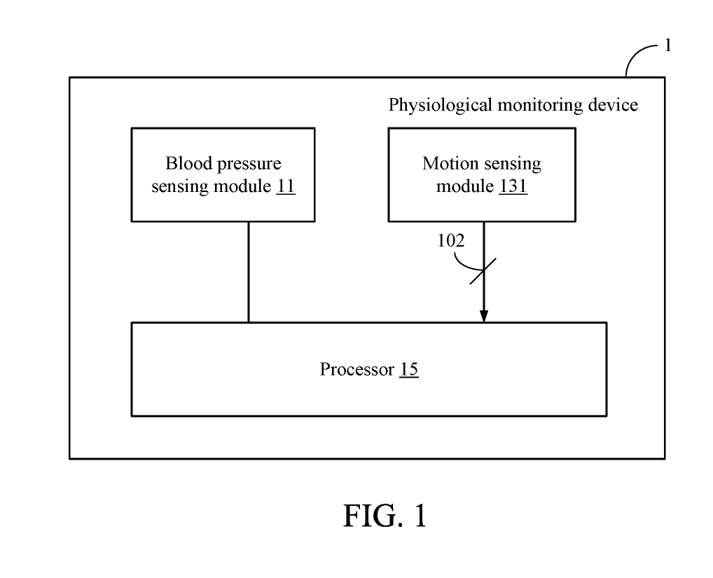

FIG. 1 depicts an exemplary embodiment of the present invention, which is a block diagram of the hardware infrastructure of a physiological monitoring device 1. The physiological monitoring device 1 of the present invention is configured to monitor the physiological state of a user, and may be a smart watch, a smart bracelet/band or any other electronic apparatus that has the physiological monitoring function of the present invention. The physiological monitoring device 1 comprises a blood pressure sensing module 11, a motion sensing module 131, and a processor 15. The processor 15 is coupled to the blood pressure sensing module 11 and the motion sensing module 131.

The blood pressure sensing module 11 can measure the blood pressure of the user with a non-invasive blood pressure measurement method, such as a cuff-less non-invasive blood pressure measurement method that can calculate the blood pressure through using sensing results of a photoplethysmographic (PPG) sensor and an electrocardiography (ECG) sensor, or with other sensors that can measure/help to calculate the blood pressure.

The motion sensing module 131 is configured to sense the physical state of the physiological monitoring device 1 to generate a physical state signal 102. The motion sensing module 131 is, for example, a 3-axis accelerometer, a micro electromechanical accelerometer, a gyroscope, a gravity sensor (G-sensor), a magnetometer or a combination thereof, or is any other inertia sensor that can be used in measuring the movement of an object.

In this exemplary embodiment, the processor 15 may determine whether the physiological monitoring device is in a stationary state and a horizontal state according to the physical state signal 102, and control the blood pressure sensing module 11 to measure a blood pressure of the user when it is determined that the physiological monitoring device is in the stationary state and the horizontal state. As an example, in a case where the motion sensing module 131 consists of a gravity sensor, the direction of the physiological monitoring device when the acceleration sensed by the gravity sensor is oriented to a certain axis is defined as the horizontal direction according to the factory settings of horizontal calibration due to the Earth's gravitational acceleration towards the ground.

Therefore, in this exemplary embodiment, the processor 15 needs to determine whether there is substantially only the gravitational acceleration at the moment and the gravitational acceleration component is oriented substantially towards a predetermined axial direction according to the received physical state signal 102 to determine whether the physiological monitoring device is in the stationary state and the horizontal state. Then, when the physiological monitoring device 1 of the present invention is being worn by the user in the stationary state and the horizontal state, the processor deduces that the user's arm shall also be in the stationary state and the horizontal state, i.e., an ideal measurement condition suitable for measuring the blood pressure is met at this point; otherwise, when the processor deduces that the user's arm is in a motion state, measuring the blood pressure at this point will be prevented to ensure the precision of the blood pressure measurement.

In another exemplary embodiment, the physiological monitoring device 1 may also comprise at least one height sensing module (not shown) therein to more precisely determine that the arm is at the same height as the heart of the user. The height sensor module, which is coupled to the processor 15, is configured to generate a target height signal (not shown) corresponding to the current position of the physiological monitoring device 1. The processor 15 determines whether the physiological monitoring device 1 is within a predetermined height interval according to the target height signal to determine whether the arm of the user is currently at substantially the same height as the heart of the user, and if it is determined that they are at substantially the same height, then the ideal measurement condition is met. As an example, the height sensing module (e.g., an air pressure sensor that can be used to sense the relative altitude of a target) is configured to sense a target air pressure value corresponding to the current position of the physiological monitoring device 1. In this example, the physiological monitoring device 1 may be placed at the same height as the heart of the user in advance for calibration purposes to record a reference air pressure value corresponding to the position of the user's heart. The processor 15 may decide the predetermined height interval according to this reference air pressure value. In this manner, the current position of the physiological monitoring device 1 that is within a predetermined height interval can be determined by the processor 15 according to the target air pressure value.

In another example, another air pressure sensor may be provided at the position of the user's heart. This air pressure sensor is configured to sense the reference air pressure value at the position of the user's heart, and transmits the measured reference air pressure value back to the physiological monitoring device 1 through a near field wireless transmission technology (e.g., Bluetooth). Then, the processor 15 determines whether the measured target air pressure value and the reference air pressure value are substantially the same (i.e., there is an allowable range for the difference between the two values) to determine whether the arm of the user is at substantially the same height as the heart of the user, thus, determining whether the ideal measurement condition is met.

The height sensing module may also be a micro electromechanical system (MEMS) pressure sensor, a pressure micro sensor, a piezoelectric pressure micro-sensor, a capacitance pressure micro-sensor, a digital air pressure sensor or a combination thereof, or be any other sensor that can be used to measure an air pressure at a position, but is not limited thereto.

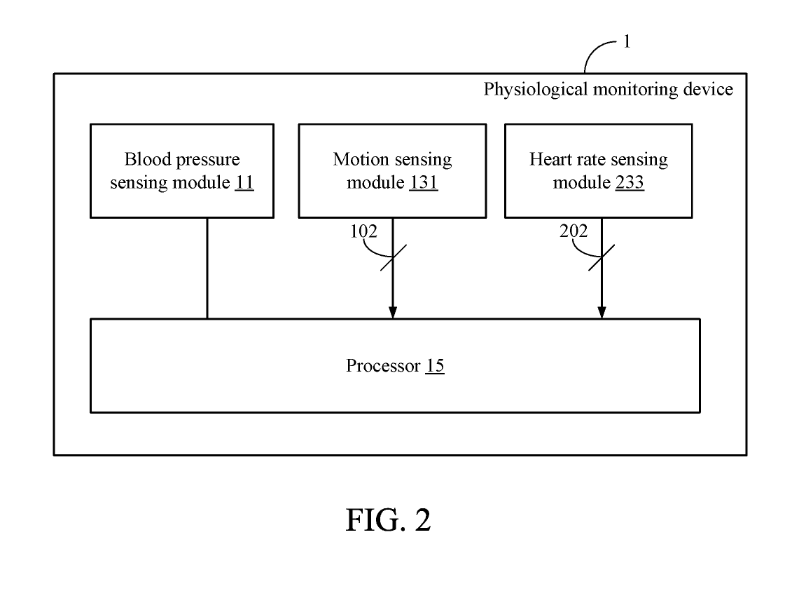

Another exemplary embodiment of the present invention is depicted in FIG. 2, which is an extensive exemplary embodiment. As compared with FIG. 1, the physiological monitoring device 1 in FIG. 2 further comprises a heart rate sensing module 233 coupled to the processor 15. The heart rate sensing module 233 is configured to sense a heart rate state of a user to generate a heart rate signal 202, and is, for example but not limited to, a photoplethysmographic sensor (PPG sensor) or an electrocardiography sensor (ECG sensor).

In this embodiment, the processor 15 further receives the heart rate signal 202 from the heart rate sensing module 233 to determine whether the heart rate state of the user is within a predetermined heart rate interval. In detail, in this embodiment, an ideal measurement condition for the processor 15 to trigger the blood pressure sensing module 11 to measure an blood pressure of the user is set to comprise: (1) the physiological monitoring device is in a stationary state and a horizontal state, and (2) the heart rate state of the user is within a predetermined heart rate interval. Therefore, when the ideal measurement condition is met, the processor 15 determines that the user is in an ideal measurement state and then triggers the blood pressure sensing module 11 to measure the blood pressure of the user.

As described above, the present invention improves the reference value of the blood pressure measurement result by determining whether the user is in the ideal measurement state to measure a blood pressure of the user when the user is in the ideal measurement state. Accordingly, apart from considering “whether the arm of the user is in the stationary state and the horizontal state”, this embodiment further takes “whether the heart rate of the user is within the predetermined heart rate interval” into consideration as compared to the previous embodiment to avoid measuring the blood pressure of the user when the user is in an emotional state or in a post-strenuous-exercise state.

It shall be appreciated that the aforesaid “predetermined heart rate interval” may be predetermined depending on other related information, for example but not limited to, the age, the gender or the like of the user.

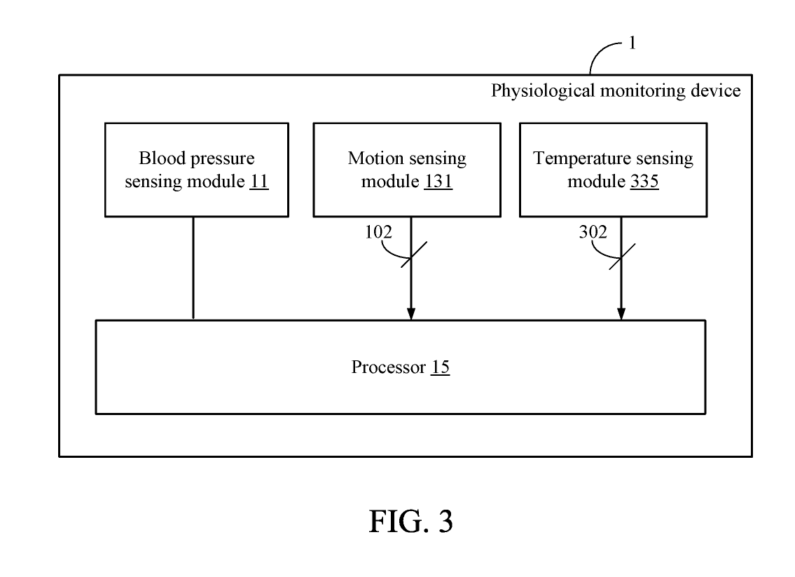

Another exemplary embodiment of the present invention is depicted in FIG. 3, which is an extensive exemplary embodiment. As compared with FIG. 1, the physiological monitoring device 1 further comprises a temperature sensing module 335. The temperature sensing module 335 may be but is not limited to an infrared temperature sensor, a thermocouple, a resistance temperature detector or the like. The temperature sensing module 335, which is coupled to the processor 15, is configured to sense the surface temperature of the user to generate a body temperature signal 302. Accordingly, the processor 15 can further receive the body temperature signal 302 from the temperature sensing module 335 and determine whether the surface temperature of the user is within a predetermined body temperature interval according to the body temperature signal 302.

In detail, in this embodiment, an ideal measurement condition for the processor 15 to trigger the blood pressure sensing module 11 to measure an blood pressure of the user is set to comprise: (1) the physiological monitoring device is in a stationary state and a horizontal state, and (2) the surface temperature of the user is within the predetermined body temperature interval. Therefore, when the ideal measurement condition is met, the processor 15 determines that the user is in an ideal measurement state and then triggers the blood pressure sensing module 11 to measure the blood pressure of the user.

Apart from considering “whether the arm of the user is in the stationary state and the horizontal state”, this embodiment further takes “whether the surface temperature of the user is within the predetermined body temperature interval” into consideration as compared to the previous embodiment to avoid measuring the blood pressure of the user when the user's body temperature is unduly high or low (e.g., when he/she has just come out of a hot/cold bath).

It shall be appreciated that the aforesaid “predetermined body temperature interval” may depend on the age and the gender input by the user (since the reference value of the body temperature varies slightly for users of different ages and genders) or depend on related information such as historical body temperature measurement records of the user, and accordingly, a corresponding “predetermined body temperature interval” is selected to assist in determining whether the ideal measurement condition is met, but the present invention is not limited thereto.

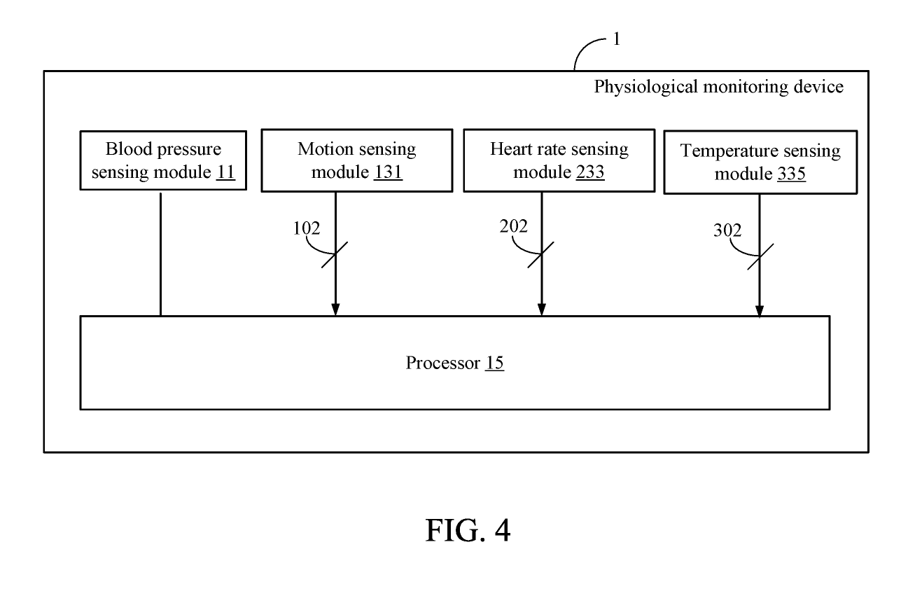

Another exemplary embodiment of the present invention is depicted in FIG. 4, which is an extensive exemplary embodiment. In this embodiment, the physiological monitoring device 1 comprises a motion sensing module 131, a heart rate sensing module 233 and a temperature sensing module 335. An ideal measurement condition of this embodiment comprises: (1) the physiological monitoring device 1 is in a stationary state and a horizontal state, (2) the heart rate state of the user is within a predetermined heart rate interval; and (3) the surface temperature of the user is within a predetermined body temperature interval. In other words, the processor 15 will determine whether the ideal measurement condition is met according to the physical state signal 102, the heart rate signal 202 and the body temperature signal 302 currently received. Accordingly, the physiological monitoring device 1 measures a blood pressure of the user when the ideal measurement condition is met.

In another embodiment, in the case where the physiological monitoring device 1 also comprises the motion sensing module 131, the heart rate sensing module 233 and the temperature 335, the ideal measurement condition may only comprise one of or any combination of the physiological monitoring device 1 being in a stationary state and a horizontal state, a heart rate of the user being within a predetermined heart rate interval, and a surface temperature of the user being within a predetermined body temperature interval. The present invention has no limitation on what comprised in the ideal measurement condition.

The physiological monitoring device 1 of each of the embodiments further comprises a timer (not shown) and an output module (not shown). The timer and the output module are both coupled to the processor 15. The timer is, for example, a system clock, an oscillator clock, an electronic timer, or any device that can provide the processor 15 with time information. The output module is, for example, a screen, a speaker, a vibrator or any combination thereof that can output a notification message. The notification message is, for example, a visual signal, a sound signal, a vibration or any combination thereof.

In this embodiment, the processor 15 needs to determine whether the physiological monitoring device 1 meets the ideal measurement condition at multiple different time points indicated by the timer (e.g., times points such as 8 o'clock, 9 o'clock, 10 o'clock, and the like) respectively according to a time period (e.g., every other hour), and controls the blood pressure sensing module 11 to measure the blood pressure when the ideal measurement condition is met.

Otherwise, when the ideal measurement condition is not met at least one of the time points, the processor 15 continuously determines whether the ideal measurement condition is met within a predetermined period and, when the ideal measurement condition is met, controls the blood pressure sensing module 11 to measure the blood pressure. When it is determined that the ideal measurement pressure is not met, the processor 15 controls the output module to output a notification message which notifies the user of adjusting his/her body posture and/or stopping his/her current activity, thereby allowing the physiological monitoring device 1 to be in the aforesaid ideal measurement state for measuring the blood pressure.

In another embodiment, the processor 15 may determine whether the ideal measurement condition is met only within a predetermined period after each blood pressure measurement without the need of measuring a blood pressure at multiple different time points respectively according to a time period. If the ideal measurement is met, then measuring the blood pressure of the user; and if the ideal measurement is not met, then the output module is controlled to output the aforesaid notification message.

In this embodiment, the physiological monitoring device 1 of the present invention may specifically track the blood pressure of the user when he/she wakes up and before he/she falls asleep to further analyze the blood pressure trend of the user effectively. In detail, the processor 15 may determine a sleep start time, a sleep end time or a sleep time of the user according to at least one of the physical state signal 102, the heart rate signal 202 and the body temperature signal 302 received within a certain time duration.

As an example, the processor 15 may determine via the motion sensing module 131 that the physiological monitoring device 1 is in the stationary state and the horizontal state for a long period of time and thus, deduce that the user is in a sleep state. The processor may determine via the heart rate sensing module 233 that the user shows a relatively low and stable heart beat for a long period of time and thus deduce that the user is in the sleep state. Furthermore, the processor 15 may determine via the temperature sensing module 335 that the user shows a relatively low and stable body temperature for a long period of time and thus, deduce that the user is in the sleep state.

In other words, the blood pressure sensing module 11 is triggered by the processor 15 to measure the user's blood pressure in the ideal measurement state when it is deduced that the user has just went into a sleep state. It shall be appreciated that, in addition to the motion sensing module 131, the time information provided by the timer may also be used to assist the physiological monitoring device 1 of the present invention in confirming whether the user is in the sleep state (e.g., determining whether it is at a night time and at a late night time).

Since the wake-up blood pressure and pre-asleep blood pressure of the user are of considerable referential importance, the physiological monitoring device 1 of the present invention may periodically measure the wake-up blood pressure and pre-asleep blood pressure of the user everyday according to the obtained sleep start time, the obtained sleep end time and the obtained sleep time so as to provide the user information about a long-term blood pressure trend. Moreover, the processor 15 determines whether the aforesaid ideal measurement condition is met within a predetermined period after the sleep end time, and controls the blood pressure sensing module 11 to measure the blood pressure when the aforesaid ideal measurement condition is met.

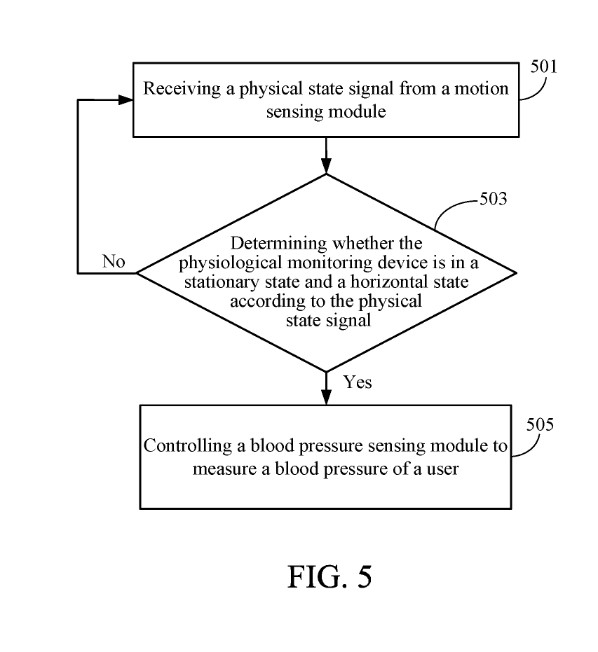

Another exemplary embodiment of the present invention is a physiological monitoring method; a flowchart diagram of which is illustrated in FIG. 5. The physiological monitoring method is applied to an electronic apparatus, e.g. the aforesaid physiological monitoring device 1 of FIG. 1.

First, in step 501, a physical state signal 102 is received from a motion sensing module 131. Then, in step 503, the processor 15 determines whether the physiological monitoring device 1 is in a stationary state and a horizontal state according to the physical state signal 102 to determine whether an ideal measurement condition is met. If the ideal measurement condition is met, then step 505 is executed to control the blood sensing module 11 to measure a blood pressure. Otherwise, the processor 15 returns to step 501 to receive a new subsequent physical state signal 102 from the motion sensing module 131.

In another embodiment, the physiological monitoring method may further comprise the following steps: receiving at least one air pressure value; and determining whether the ideal measurement condition is met by determining whether an arm of the user is currently at the same height as the heart of the user according to the at least one air pressure value.

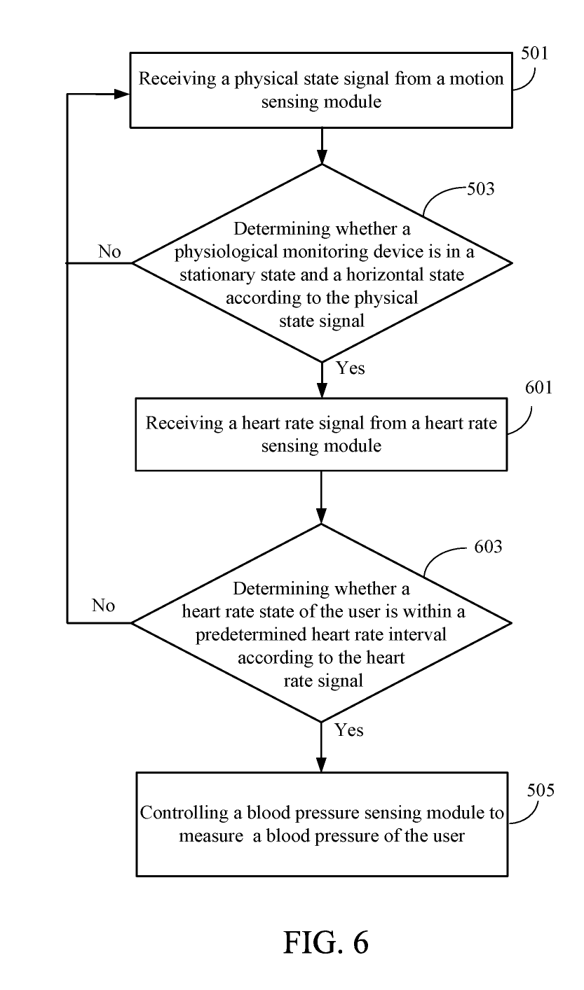

Another exemplary embodiment of the present invention is a physiological monitoring method; a flowchart diagram of which is illustrated in FIG. 6. As compared with FIG. 5, the physiological monitoring method is applied to an electronic apparatus like the aforesaid physiological monitoring device 1 of FIG. 2. After it is determined that the physiological monitoring device 1 is in a stationary state and a horizontal state in step 503, step 601 is further executed to receive the heart rate signal 202 from the heart rate sensing module 233. Then, step 603 is executed to determine whether the heart rate state of the user is within a predetermined interval according to the heart rate signal 202. If the result of the step 603 is “Yes”, then step 505 is executed. If the result of the step 603 is “No”, then the process returns to step 501. In other words, the ideal measurement condition in this embodiment comprises: the physiological monitoring device 1 is in the stationary state and the horizontal state, and the heart rate state of the user is within the predetermined heart rate interval. It shall be appreciated that in some embodiments, step 601 and step 501 may also be executed simultaneously, and step 503 and step 603 may also be executed simultaneously as well.

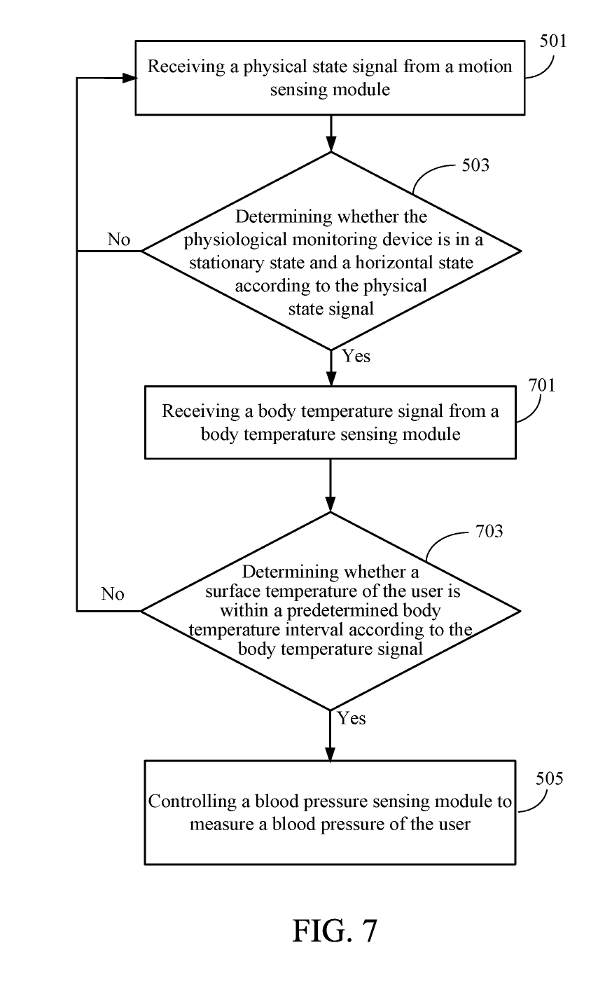

Another exemplary embodiment of the present invention is a physiological monitoring method, a flowchart diagram of which is illustrated in FIG. 7. As compared with FIG. 5, the physiological monitoring method is applied to an electronic apparatus like the aforesaid physiological monitoring device 1 of FIG. 3. After it is determined that the physiological monitoring device 1 is in a stationary state and a horizontal state in the step 503, step 701 is further executed to receive a body temperature signal 302 from a temperature sensing module 335. Then, step 703 is executed to determine whether the surface temperature of the user is within a predetermined body temperature interval according to the body temperature signal 302. If the result of the step 703 is “Yes”, then the step 505 is executed. If the result of the step 703 is “No”, then the process returns to the step 501. In other words, the ideal measurement condition in this embodiment comprises: the physiological monitoring device 1 is in the stationary state and the horizontal state, and the surface temperature of the user is within the predetermined body temperature interval. It shall be appreciated that, in some embodiments, the step 501 and the step 701 may also be executed simultaneously, and the step 503 and the step 703 may also be executed simultaneously as well.

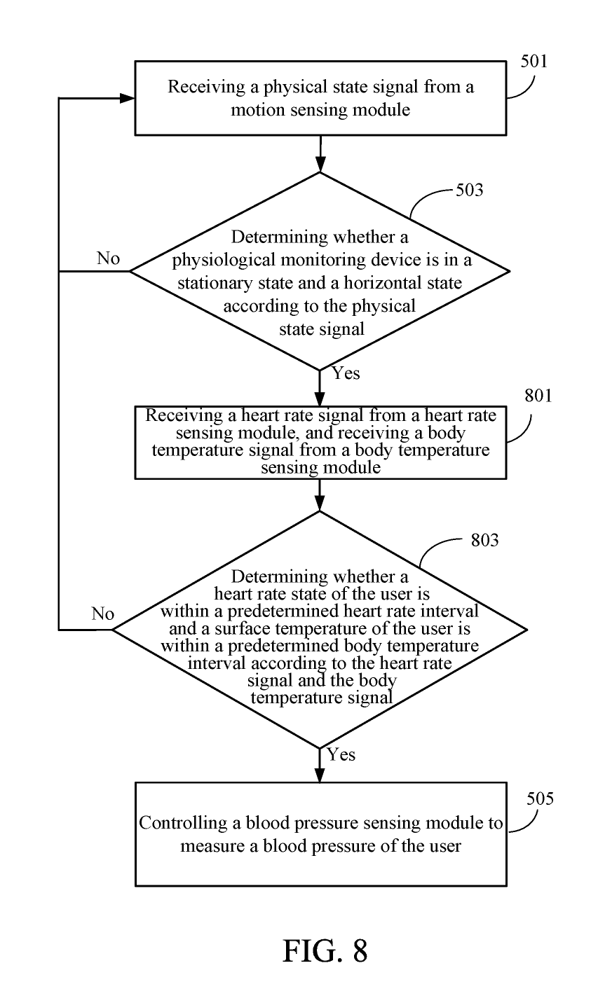

Another exemplary embodiment of the present invention is a physiological monitoring method, a flowchart diagram of which is illustrated in FIG. 8. As compared with FIG. 5, the physiological monitoring method is applied to an electronic apparatus like the aforesaid physiological monitoring device 1 of FIG. 4. After it is determined that the physiological monitoring device 1 is in a stationary state and a horizontal state in the step 503, step 801 is executed to receive a heart rate signal 202 from a hear rate sensing module 233 and receive a body temperature signal from a temperature sensing module 335. Then step 803 is executed to determine whether a heart rate state of a user is within a predetermined heart rate interval and whether a surface temperature of the user is within a predetermined body temperature interval according to the heart rate signal 202 and the body temperature signal 302. If the result of the step 803 is “Yes”, then the step 505 is executed to control a blood pressure sensing module 11 to measure a blood pressure. If the result of the step 803 is “No”, then the process returns to the step 501. In other words, the ideal measurement condition in this embodiment comprises: the physiological monitoring device 1 is in the stationary state and the horizontal state, the heart rate state of the user is within the predetermined heart rate interval, and the surface temperature of the user is within the predetermined body temperature interval. It shall be appreciated that, in some embodiments, the step 501 and the step 801 may also be executed simultaneously, and the step 503 and the step 803 may also be executed simultaneously as well.

Details of the individual steps in each aforesaid physiological monitoring method have been given in the aforesaid exemplary embodiments of FIG. 1 to FIG. 4, and thus will not be further described herein. How the present embodiment executes these operations and have these functions will be readily appreciated by those of ordinary skill in the art based on the explanation of the aforesaid embodiments, and thus will not be further described herein.

It shall be appreciated that, the aforesaid physiological monitoring method of the present invention may be implemented by a non-transitory computer readable storage medium. In particular, after a computer program recorded in the non-transitory computer readable storage medium is loaded into and installed in an electronic apparatus, the instructions comprised in the computer program are executed by a processor of the electronic apparatus to execute the physiological monitoring method of the present invention. The non-transitory computer readable storage medium may be a read only memory (ROM), a flash memory, a floppy disk, a hard disk, a compact disk (CD), a mobile disk, a magnetic tape, a database accessible to networks, or any other storage with the same function and well known to those skilled in the art. In an implementation, the computer program can be downloaded by the user via a network. In another implementation, the computer program has already been built in the electronic apparatus.

In summary, the physiological monitoring mechanism of the present invention can monitor a physiological condition of a user in real time so as to ensure that measuring a blood pressure of the user when the user is in an ideal measurement condition. Besides, a long-time automatic tracking of a pre-asleep blood pressure and a wake-up blood pressure of the user can also be made by the physiological monitoring mechanism of the present invention. Accordingly, the physiological monitoring mechanism of the present invention can not only ensure that all the blood pressure values measured are measured when the user is in an ideal measurement state so as to improve effectiveness and the reference value of the blood pressure measurement result, but also assist the user in analyzing his/her long-term blood pressure trend (including a predetermined period, a time period, the pre-asleep blood pressure and the wake-up blood pressure) so as to provide the user with his/her own physiological tracked information of a great reference value.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A physiological monitoring device, comprising:
a blood pressure sensing module;
a motion sensing module configured to sense a physical state of the physiological monitoring device to generate a physical state signal; and
a processor coupled to the blood pressure sensing module and the motion sensing module, and configured to determine whether the physiological monitoring device is in a stationary state and a horizontal state according to the physical state signal so as to determine whether an ideal measurement condition is met, and control the blood pressure sensing module to measure a blood pressure of a user when the ideal measurement condition is met.

2. The physiological monitoring device of claim 1, further comprising:
a height sensing module coupled to the processor and configured to generate a target height signal corresponding to the physiological monitoring device;
wherein the processor further determines whether the physiological monitoring device is within a predetermined height interval according to the target height signal so as to determine whether the ideal measurement condition is met.

3. The physiological monitoring device of claim 1, further comprising:
a heart rate sensing module coupled to the processor and configured to generate a heart rate signal by sensing a heart rate state of the user;
wherein the processor further determines whether the heart rate state is within a predetermined heart rate interval according to the heart rate signal so as to determine whether the ideal measurement condition is met.

4. The physiological monitoring device of claim 1, further comprising:
a temperature sensing module coupled to the processor and configured to generate a body temperature signal by sensing a surface temperature of the user;
wherein the processor further determines whether the surface temperature of the user is within a predetermined body temperature interval according to the body temperature signal so as to determine whether the ideal measurement condition is met.

5. The physiological monitoring device of claim 1, further comprising:
a heart rate sensing module coupled to the processor and configured to generate a heart rate signal by sensing a heart rate state of the user; and
a temperature sensing module coupled to the processor and configured to generate a body temperature signal by sensing a surface temperature of the user;
wherein the processor further determines whether the heart rate state of the user is within a predetermined heart rate interval according to the heart rate signal and whether the surface temperature of the user is within a predetermined body temperature interval according to the body temperature signal so as to determine whether the ideal measurement condition is met.

6. The physiological monitoring device of claim 5, wherein the processor further determines a sleep start time, a sleep end time, or a sleep time of the user according to at least one of the physical state signal, the heart rate signal, and the body temperature signal.

7. The physiological monitoring device of claim 6, wherein the processor further determines whether the ideal measurement condition is met within a predetermined period after the sleep end time, and controls the blood pressure sensing module to measure the blood pressure of the user when the ideal measurement condition is met.

8. The physiological monitoring device according to claim 1, further comprising:
a timer coupled to the processor, wherein after measuring the blood pressure of the user, the processor further determines whether the ideal measurement condition is met after a predetermined period, and controls the blood pressure sensing module to measure a blood pressure of the user when the ideal measurement condition is met.

9. The physiological monitoring device according to claim 8, wherein the processor generates a notification message after the predetermined period, when the processor determines that the ideal measurement condition is not met.

10. A physiological monitoring method, comprising:
(a) receiving a physical state signal corresponding to the electronic apparatus;
(b) determining whether the electronic apparatus is in a stationary state and a horizontal state according to the physical state signal so as to determine whether an ideal measurement condition is met; and
(c) controlling the electronic apparatus to measure a blood pressure of a user when the ideal measurement condition is met.

11. The physiological monitoring method of claim 10, further comprising:
receiving a target height signal corresponding to the electronic apparatus; and
further determining whether the electronic apparatus is within a predetermined height interval according to the target height signal so as to determine whether the ideal measurement condition is met.

12. The physiological monitoring method of claim 10, further comprising:
receiving a heart rate signal corresponding to the user; and
further determining whether a heart rate state of the user is within a predetermined heart rate interval according to the heart rate signal so as to determine whether the ideal measurement condition is met.

13. The physiological monitoring method of claim 10, further comprising:
receiving a body temperature signal corresponding to the user; and
further determining whether a surface temperature of the user is within a predetermined body temperature interval according to the body temperature signal so as to determine whether the ideal measurement condition is met.

14. The physiological monitoring method of claim 10, further comprising:
receiving a heart rate signal and a body temperature signal corresponding to the user; and
further determining whether a heart rate state of the user is within a predetermined heart rate interval according to the heart rate signal and whether a surface temperature of the user is within a predetermined body temperature interval according to the body temperature signal so as to determine whether the ideal measurement condition is met.

15. The physiological monitoring method of claim 14, further comprising:
determining a sleep start time, a sleep end time, or a sleep time of the user according to at least one of the physical state signal, the heart rate signal, and the body temperature signal.

16. The physiological monitoring method of claim 15, further comprising:
determining whether the ideal measurement condition is met within a predetermined period after the sleep end time; and
controlling the electronic apparatus to measure the blood pressure of the user when the ideal measurement condition is met.

17. The physiological monitoring method according to any one of claims 10, further comprising:
determining whether the ideal measurement condition is met within a predetermined period after measuring the blood pressure of the user; and
controlling the electronic apparatus to measure the blood pressure of the user when the ideal measurement condition is met.

18. The physiological monitoring method according to any one of claims 17, further comprises:
controlling the electronic apparatus to generate a notification message when it is determined that the ideal measurement condition is not met within the predetermined period after measuring the blood pressure of the user.

19. A non-transitory computer readable storage medium, having a computer program stored therein, the computer program being loaded into an electronic apparatus to execute the following steps of:
(a) receiving a physical state signal corresponding to the electronic apparatus;
(b) determining whether the electronic apparatus is in a stationary state and a horizontal state according to the physical state signal so as to determine whether an ideal measurement condition is met; and
(c) controlling the electronic apparatus to measure a blood pressure of a user when the ideal measurement condition is met.
